(19) United States
(12) Patent Application Publication
(10) Pub. No.: US 2009/0192548 A1
(43) Pub. Date: Jul. 30, 2009

(54) Pedicle-laminar dynamic spinal stabilization device

(76) Inventors: Dong M. Jeon, Draper, UT (US); Patrick D. Moore, West Jordan, UT (US); Hee J. Yang, Sungnam-si (KR); Sang K. Lee, Seoul (KR)

Correspondence Address: MORRISS OBRYANT COMPAGNI, P.C., 734 EAST 200 SOUTH, SALT LAKE CITY, UT, 84102, US

(21) Appl. No.: 12/321,971

(22) Filed: Jan. 26, 2009

Related U.S. Application Data
(60) Provisional application No. 61/023,784, filed on Jan. 25, 2008.

(51) Int. Cl. A61B 17/70; A61B 17/58

(52) U.S. Cl. 606/246; 606/264; 606/278; 606/301; 606/257

(57) ABSTRACT

Apparatus, systems and methods for decompression and dynamic stabilization of the vertebrae. A flexion construct is formed from a medial loop of a cylindrical member in a first plane that is generally parallel to the long axis of the member, and two outlying loops, generally residing in a common second plane that is generally parallel to the long axis of the member and generally perpendicular to the first plane formed by the medial loop. The medial loop and outlying loops of the flexion construct may aid in controlling distraction, compression and flexion of the assembly. Multiple level constructs may include multiple sets of loops, one set for each spinal level. The member may also include two legs that terminate in hooks or include a straight portion for attachment by another attachment means. A protective sleeve may be used between the member and any attachment means.

### CROSS-REFERENCE TO RELATED APPLICATION

This application claims the benefit of U.S. Provisional Application No. 61/023,784, filed Jan. 25, 2008, the disclosure of which is incorporated herein by reference in its entirety.

### TECHNICAL FIELD

The present invention relates to devices and implants used in osteosynthesis and other orthopedic surgical procedures such as devices for use in spinal surgery, and, in particular, to orthopedic stabilization devices used to limit the relative motion of at least two vertebral bodies for the relief of pain. These devices can be used to aid osteo-synthesis in combination with fusion devices, supplement other motion restoring devices such as disk implants, or used solely to restrict the motion of vertebral bodies as stand-alone devices.

### BACKGROUND

There have been many devices contrived to relieve pain associated with spinal injury or illness. Traditionally surgeons have fused the vertebral bodies with a pedicle screw and solid rod construct or a fusion cage. In attempting to fuse the spine using traditional methods, patients may experience a long and painful recovery process as well as the uncertainty of the formation mass. Most rod and screw constructs and fusion cage constructs are very rigid, not allowing transfer of stress into the fusion site that would otherwise aid in a quicker recovery and promote the boney fusion mass. It is well known, that where stress is allowed to transfer through the fusion site while the vertebral bodies are held in a limited range of motion, then fusion can occur much quicker aiding in patient recovery time.

There are many devices that allow relative motion, yet these have fallen short in preventing the shear forces between the vertebral bodies being stabilized. Another shortcoming is that relative motion has been forcibly channeled through a rather specific location or hinge point in the mechanical construct. The following discussion more particularly summarizes some of these efforts.

U.S. Pat. No. 5,092,866, the disclosure of which is incorporated herein by reference in its entirety, describes a pedicle screw system that is banded together with flexible ligaments. While the ligaments allow for relative motion, they do not appear to resist compression or shear loads, instead relying upon tension alone.

European Patent No. EP 06691091 A1/B1, the disclosure of which is incorporated herein by reference in its entirety, describes a polycarbonate/urethane supporting element compressed between two adjacent pedicle screws and passing over an elastic strap that acts as a flexible internal ligament. This flexible internal ligament is in the form of a nylon cord, which is pre-tensioned and fastened to the screw heads. This design provides flexural degrees of freedom and allows relative motion between the vertebral bodies, but does little to inhibit or prevent shearing between the vertebral bodies. While flexibility is desirable, this type of ligament appears to lack rigidity and rely on proper tensioning inter-operatively to gain its balance.

U.S. Pat. No. 6,267,764, the disclosure of which is incorporated herein by reference in its entirety, discloses a pedicle screw and rod system wherein the rod is flexible in translation. A dampening ball is not separate from the rods and has cutouts to allow bending, with no ligament passing through the centers of the rods. While flexibility in translation can be helpful, the spine loads in several planes at the same time and the translation spoken of in this patent would appear to inadequately redistribute stresses through the fusion site. As a result, motion is forcibly limited to one location, i.e., motion is constrained through a hinge point, which undesirably stresses the assembly construct.

Accordingly, there exists a need for assemblies and devices that effectively resist torsion as well as shear forces while providing flexible stabilization. More specifically, it would be desirable to provide kits with such assemblies and devices, which work with existing pedicle screw arrangements if required.

There is a further need yet to provide a stabilization device that can allow natural flexion and extension motion while effectively restraining torsional and shear forces.

There is a further need to provide stabilization assemblies and devices manufactured from a shape memory material such as an alloy or other flexible polymer, which can withstand repeated loading of the spine without fatiguing yet still maintain its flexibility.

### SUMMARY

Apparatus, systems and methods for decompression and dynamic stabilization of the vertebrae. A cylindrical member has a spinal level flexion construct formed from a medial loop in a first plane and two formed in a first plane that is generally parallel to the long axis of the member, and defined by the portion of the member forming the medial loop, and two outlying loops, each characterized as a looped section of the member formed at a side of the medial loop and between the medial loop and an end of the member and generally residing in a common second plane that is generally parallel to the long axis of the member and generally perpendicular to the first plane formed by the medial loop. The medial loop and outlying loops of the member aid in controlling distraction, compression and flexion of the assembly upon installation. For multiple level constructs, multiple sets of medial and outlying loops may be used, one set for each spinal level. The member continues past the flexion portion to two respective ends to form two legs. The legs may terminate in hooks for installation by hooking over vertebral lamina or pedicles, or may lack hooks and include a straight portion for attachment by screws or other attachment means. Where screws are used, a sleeve may be placed over the portion of the leg to provide an interface between the screw and the member.

For installation, two of each such resiliently compressible members that are adapted for position between adjacent superior and inferior vertebrae may be attached thereto, on upon each side of the spine, and positioned to provide dynamic decompression and stabilization to the adjacent vertebral bodies. Without being bound by any theory, it is believed that the utilization of such apparatus and systems may provide intervertebral disk distraction and stabilization forces to result in restoring of at least some natural spinal segment placement, which alleviates compression on the intervertebral disk and stabilizes the adjacent spinal segments, allowing the systems to be used on a standalone basis or as an adjunct to spinal fusion.

### DESCRIPTION OF THE DRAWINGS

It will be appreciated by those of ordinary skill in the art that the elements depicted in the various drawings are not necessarily to scale, but are for illustrative purposes only. The nature of the present invention, as well as other embodiments of the present invention may be more clearly understood by reference to the following detailed description of the invention, to the appended claims, and to the several drawings attached hereto.

FIG. 1 is a diagram of the shape memory of Nitinol components.

FIG. 2 is a diagram of a loading/unloading curve for Nitinol.

FIGS. 3A and 3B generally depict top and side views of a first embodiment of a Pedicle-Laminar Dynamic Spinal Stabilization device assembly 10 which is a single-level spine construct installed in two bone screws in accordance with the principles of the present invention.

FIG. 4 shows a sectional side view of the embodiment of FIGS. 3A and 3B, through a bone screw.

FIGS. 5A and 5B depict top and side views of the embodiment of FIGS. 3A and 3B without the bone screws.

FIGS. 6A and 6B depict top and side views of a two-level construct in accordance with the present invention.

FIG. 6C depicts a two-level construct, where one level is for use in a spinal fusion procedure.

FIGS. 7A and 7B depict top and side views of a three-level construct in accordance with the present invention.

FIG. 8 depicts a side view of the sleeve member of FIG. 4.

FIG. 9 depicts various hook conformations for embodiments in accordance with the present invention.

### DETAILED DESCRIPTION

For the purposes of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

Dynamic stabilization of damaged or diseased spinal segments has long been desired. However, until recently, the technology has yet been underdeveloped. Numerous techniques and devices have been developed with varying degrees of success. These dynamic stabilization applications include flexible rod systems, Interspinous Process Decompression devices and artificial disks. These different systems are successful in some aspects and failures in others, as well as being indicated for a wide variety of uses; however, no device is all inclusive for all indications. Some failures of these known devices can be attributed to the devices' material of manufacture. By nature, dynamic stabilization requires movement in the device. These devices utilize relatively static materials for construction, and therefore lack inherent dynamic material qualities.

In one embodiment, systems in accordance with the present invention utilize Nitinol for the rod and Titanium or any suitable compatible material sleeve components. Below is a summary of Nitinol's unique portfolio of properties together with several applications. The examples and discussions here are provided only to illustrate.

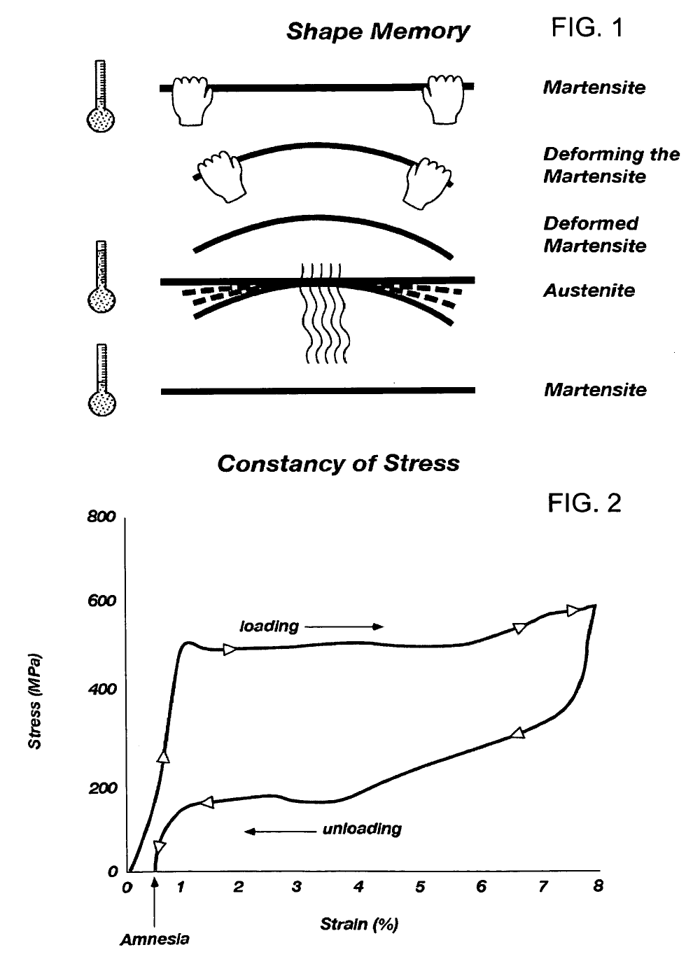

Nitinol-based products have been on the market since the late 1960's. Nitinol possesses thermal shape memory behavior. Chilling a Nitinol component converts the Austenite structure of the Nitinol to a Martensite structure, becoming very malleable. Where the chilled component is then heated, the Martensite structure of the Nitinol returns to an Austenite structure and, thus, reverts the component to its original shape, as illustrated in the diagram shown in FIG. 1. Thus, in the medical device industry, Nitinol has been used for reusable medical instruments. Surgeons can shape an instrument on site to fit a patient's geometry, then after heat sterilization the device returns to its original shape for the next procedure.

In certain embodiments of the present invention, the unique thermal shape memory behavior of Nitinol may be utilized in the installation of the device. Where an embodiment in accordance with the present invention is used as a stand-alone device, that is to say, utilized without additional screw or hook attachment means, such a device may be chilled in saline, which converts the Austenite structure of the Nitinol to a Martensite structure, becoming very malleable. The surgeon then has the ability to deform the incorporated “hooks” of the device allowing easy installation at a lamina location or a pedicle location. Once installed, the surgeon may then flood the rod component with heated saline which converts the Martensite structure of the Nitinol to an Austenite structure and, thus, reverts the device to its original shape. This type of installation can be used where the embodiments in accordance with the present invention are formed of superelastic Nitinol. For such embodiments, chilling the device in a delivery system may keep the device in the soft martensite phase in a lower force state. After deployment, as the device warms to its new surroundings, it may recover its “programmed” shape and become superelastic.

Nitinol has an increased elasticity compared to stainless steel, allowing it to be bent more significantly than stainless steel without taking a set. Nitinol's elasticity or “springback” is some 10 times greater than stainless steel. Where embodiments in accordance with the present invention are formed of superelastic Nitinol, this unique property may be utilized to allow the embodiments, once installed, to be flexible without yielding under the stresses of the application. Superelastic Nitinol has an unloading curve that stays flat over large strains, thus, i.e. Nitinol devices can be designed that apply a constant stress over a wide range of shapes. FIG. 2 depicts a diagram of a loading/unloading curve for Nitinol.

Nitinol has been approved for many clinical applications including orthopedic bone anchors, vena cava filters, cardiovascular endoprostheses, and orthopedic archwires. Other Nitinol orthopedic applications include osteosynthesis staples and scoliosis correction rods. The biocompatibility of Nitinol results mainly from its tight intermetallic bounded structure, its chemically stable and homogeneous TiO₂ surface layer, and its corrosion resistance, which is similar to other Titanium alloys.

The material specification for Nitinol conforms to ASTM standard ASTM F 2063-00, which is incorporated herein by reference in its entirety. The material specification is set forth in Table 1 below.

TABLE 1
Material Specification for Nitinol

| | Weight |
|---|---|
| Element | Percent |
| Nickel | 54.5~57 |
| Carbon, Max. | 0.070 |
| Cobalt, Max. | 0.050 |
| Copper, Max. | 0.010 |
| Chromium, Max. | 0.010 |
| Hydrogen, Max. | 0.005 |
| Iron, Max. | 0.050 |
| Niobium, Max. | 0.025 |
| Oxygen, Max. | 0.050 |
| Titanium | balance |

Although illustrative embodiments constructed from Nitinol are primarily discussed herein, it will be appreciated that pedicle laminar dynamic stabilization devices in accordance with the present invention can be manufactured out of any suitable dynamic material, or combination of materials, including polymer and stainless steel, or polymer and titanium materials.

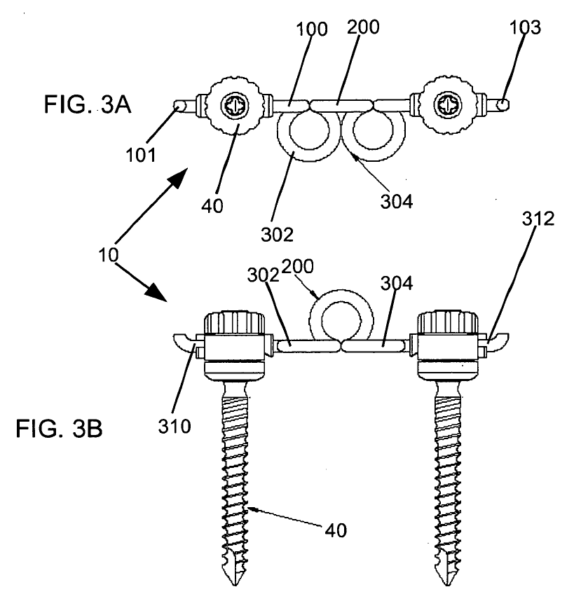

FIGS. 3A and 3B generally depict a first embodiment of a Pedicle-Laminar Dynamic Spinal Stabilization device assembly 10 in accordance with the principles of the present invention. In the illustrated embodiment, assembly 10 is formed from a member 100 having a cylindrical cross-section that extends from a first end 101 to a second end 103. The member 100 forms a medial loop structure 200 characterized as a looped section of member 100 formed at or near a midpoint between first end 101 and second end 103, with the looped section generally residing in a first plane that is generally parallel to the long axis of the member 100 and formed by the portion of member 100 forming the looped section.

The member 100 also forms two outlying loop structures 302 and 304, each characterized as a looped section of member 100 formed at or near the termination of a medial loop structure 200 and between medial loop structure 200 and the respective end 101 or 103 of the member 100. The looped section of each of outlying loop structures 302 and 304 generally resides in a second plane that is generally parallel to the long axis of the member 100 and generally perpendicular to the first plane formed by the medial loop structure 200. Medial loop structure 200 and outlying loop structures 302 and 304 aid in controlling distraction, compression and flexion of the assembly upon installation.

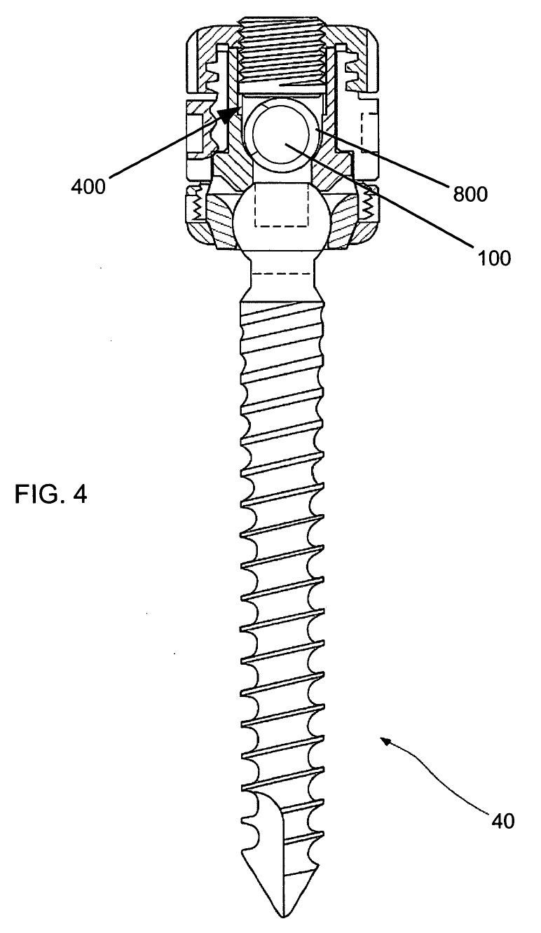

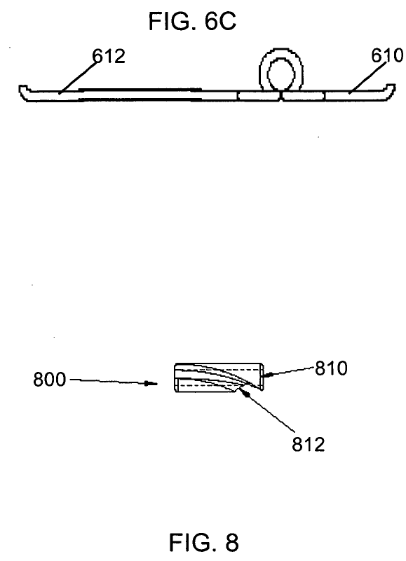

In the embodiment depicted in FIGS. 3A and 3B, the member 100 continues to extend out from the outlying loops 302 and 304 to the respective ends 101 and 103 to form two legs 310 and 312. The length of the member 100 in the legs 310 and 312 may vary based on the planned use of the assembly 10, as discussed in connection with FIG. 6C below. As depicted, in some embodiments, the legs 310 and 312 may be used for installation of the assembly 10, as by attachment to an attachment means, such as bone screws 40. As depicted in FIG. 4, this may be accomplished by securing the leg portion of member 100 in the securing channel 400 of an appropriate bone anchor assembly, which may include the use of a protective sleeve 800 for protection of the assembly 10 (as discussed further herein in connection with FIG. 8). In the depicted embodiment, the attached bone screw assemblies 40 are poly-axial pedicle screw assemblies, similar to those described in pending U.S. patent application Ser. No. 11/648,983 the disclosure of which is incorporated herein by reference in its entirety. It will be appreciated that other suitable bone anchor assemblies may be used, including poly-axial or mono-axial hooks, mono-axial or poly-axial pedicle screws, or other attachment means utilized in spinal surgery. As depicted, each of ends 101 and 103 may be bent away from the long axis of member 100.

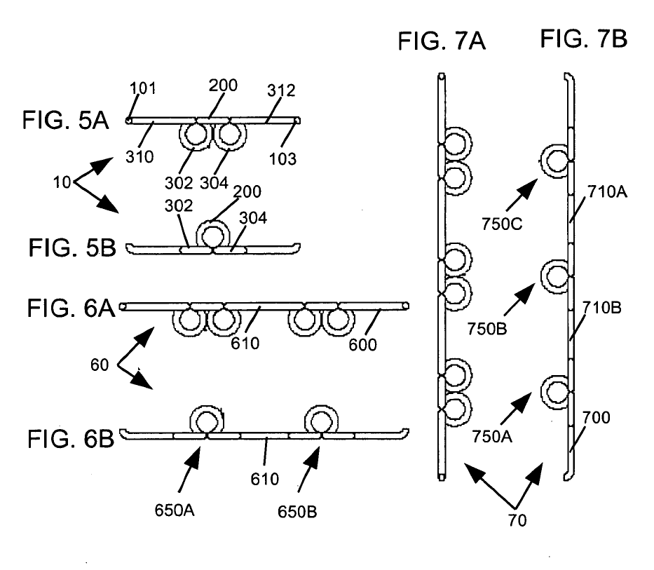

It will be appreciated that the embodiment depicted in FIGS. 3A and 3B is a single level spine construct that includes only a single set of medial and outlying loops, 200, 302 and 304. This assembly 10 is depicted in FIGS. 5A and 5B without a supplementary attachment means, such as screws 40.

FIGS. 6A and 6B depict a two level spine construct 60 that includes two sets of medial and outlying loops, 650A and 650B that are joined by an intermediate straight portion 610 of member 600. Similarly, FIGS. 7A and 7B depict a three level spine construct 70 that includes three sets of medial and outlying loops, 750A, 750B, and 750C with two intermediate straight portions 710A and 710B of member 700 therebetween. As with the single level construct of FIGS. 3A and 3B and FIGS. 5A and 5B, each construct level of these embodiments includes a medial loop member and two outlying loops to aid in controlling distraction, compression and flexion. It will be appreciated that the medial and outlying loops of each level of a construct in accordance with the present invention may generally depict a bilateral symmetry outwards from a central point of the medial loop. However, this symmetry may vary as needed for a particular application, or as the assembly is bent and shaped by a practitioner for installation in a patient.

FIG. 6C depicts a two-level construct for use in performing a spinal fusion procedure and applying the distraction and decompression benefits of the loop flexion construct at an adjacent vertebral level. One leg 612 extends a short distance, suitable for fixing the set of loops over the vertebral disk to be decompressed thereby. The second leg 610 extends further and lacks the loop structures, allowing it to be used as a rod for a spinal fusion procedure. The exact length of the second leg 610 may vary based upon the number of vertebral levels to be fused, although the depicted embodiment shows a single level length rod. For installation, the leg 610 may be placed with multiple bone anchors as the fusion is performed, and the loop-containing portion placed over the adjacent vertebral level. This may be especially useful for the treatment or prevention of adjacent disk disease.

Referring generally to FIG. 8, there is shown a sleeve member 800. This sleeve may be placed on or about the member 100 and resides as an interface between an attachment means and the device 10, as depicted in FIG. 4. Sleeve 800 may be configured as a cylinder having a bore 810 running axially throughout the sleeve (depicted in dashed lines), and a helical slot 812 extending from an outer surface thereof to the bore 810 to allow for a twisting connection onto member 100 to take place. The helical slot 812 may have a sweep angle of about 180 degrees. In a typical installation with screws 40, two sleeves may be used, one at each location for insertion in the screw for the protection of the assembly 10. It will be appreciated that the sleeve member 800 may act as a collet, clamping onto the member 100. Additionally, different sleeve members 800 with differing thicknesses may be used to allow the device 10 to be used with different anchoring means having different channel sizes.

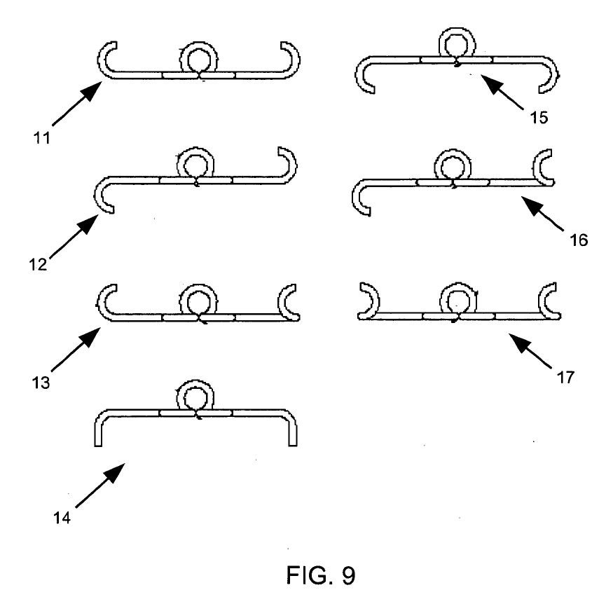

Referring generally to FIG. 9, there is shown an embodiment that includes hook geometry used as the attachment means. Such hooks may be used as hooks for attachment over the vertebral lamina or pedicle, or may be used for hook-style attachment to another suitable anatomical structure. For such embodiment, no supplementary attachment means, such as a pedicle screw, may be required. Suitable hook geometries include, but are not limited to those depicted. Assembly 11 (posterior dual convex hook configuration) illustrates a configuration with a posterior facing convex hook at either end for utilization in applying a compressive force. Assembly 12 (posterior/anterior combo convex hook configuration) illustrates a configuration with the convex hooks facing posterior and anterior and applying a compressive force. Assembly 13 (posterior convex/concave hook configuration) illustrates configuration with a convex hook and a concave hook facing posterior and applying a combined distractive/compressive force. Assembly 14 (anterior straight hook configuration) depicts straight hooks facing anterior for applying a compressive force. Assembly 15 (anterior convex hook configuration) depicts convex hooks facing anteriorly for applying a compressive force. Assembly 16 (posterior/anterior combo hook configuration) depicts a concave hook facing posterior and a convex hook facing anterior for applying a combination distractive/compressive force. Assembly 17 (posterior concave hook configuration) depicts concave hooks facing posterior for applying a distractive force.

Preferred materials for the present invention include Nitinol (NiTi). It will be recognized that any sturdy biocompatible material, such as suitable polymers or plastics, may be used to accomplish the osteosynthesis and other orthopedic surgical goals of the present invention. It will be appreciated that although a medial loop and two outlying loops are discussed herein as the illustrative spinal level flexion construct, that such shapes may be altered by a practitioner for installation and that other shapes including spring, coil, wave, and offset circle shapes may be used as needed to increase or decrease and more predictably control the flexion, extension and compression of the device.

The present invention additionally includes methods related to vertebral decompression and dynamic stabilization to provide intervertebral disk distraction and stabilization forces in an attempt to restore natural spinal segment placement, and alleviate compression on the intervertebral disk, thereby stabilizing adjacent spinal segments. This may be done as an adjunct to a spinal fusion procedure or as a standalone procedure. For such an installation, a practitioner will determine the proper size assembly 10 for use. This will be based on the number of vertebral levels affected, and a measurement of the particular patient's anatomy. For the purposes of clarity, this will be explained using a single level construct and the installation of a single assembly 10. However, it will be appreciated that in a typical surgery, two assemblies 10 will be installed, one on either side of the spine.

Where the construct is to be retained by hooking the lamina, the lamina is to be prepared and grated by laminar hook, in accordance with customary procedure. Where the construct is to be attached by other attachment means, the means is prepared, as by placement of pedicle screws at the appropriate location, such as the standard pedicle location or lamina location for such spinal fusion procedures. The selected implant assembly 10, constructed of Nitinol, is then chilled in saline, as by loading in saline of about 4 degrees C. for about 1 to 2 minutes, to convert the Austenite structure of the Nitinol to a Martensite structure. The now malleable construct may then be bent, as with a needle holder, before application. For example, a surgeon may deform the incorporated “hooks” of the device for easy installation at a lamina location or a pedicle location.

The construct is then placed in the correct position, as by attachment to an attachment means, such as bone screws, or by placement of the hooks over the grated lamina. Once installed, the surgeon may then flood the rod component with heated saline, for example saline heated to from about 40 to about 45 degrees C., to convert the Martensite structure of the Nitinol to an Austenite structure and, thus, restoring the construct to its original shape and becoming superelastic.

While the present invention has been shown and described in terms of preferred embodiments thereof, it will be understood that this invention is not limited to any particular embodiment and that changes and modifications may be made without departing from the true spirit and scope of the invention as defined and desired to be protected.

What is claimed is:

1. A dynamic spinal stabilization device, comprising:
a member having a cylindrical cross-section that extends from a first end to a second end;
a medial loop formed as a first looped section of the member, the medial loop generally residing in a first plane that is generally parallel to a long axis of the member and formed by the first looped section;
a first outlying loop formed as a second looped section of the member between the medial loop and the first end, the first outlying loop generally residing in a second plane that is generally parallel to the long axis of the member and generally perpendicular to the first plane formed by the medial loop;
a second outlying loop formed as a third looped section of the member between the medial loop and the second end, the second outlying loop generally residing in the second plane;
a first leg formed as a section of the member between the first outlying loop and the first end; and
a second leg formed as a section of the member between the second outlying loop and the second end.

2. The dynamic spinal stabilization device of claim 1, wherein the member is constructed from Nitinol.

3. The dynamic spinal stabilization device of claim 1, wherein the medial loop, first outlying loop and second outlying loop have bilateral symmetry outwards from a central point of the medial loop.

4. The dynamic spinal stabilization device of claim 1, further comprising a slotted sleeve for placement on the first leg for installation of the first leg into a bone anchor.

5. The dynamic spinal stabilization device of claim 4, wherein the slotted sleeve comprises a cylinder having a bore running axially therethrough and a helical slot extending from an outer surface thereof to the bore.

6. The dynamic spinal stabilization device of claim 1, wherein the first leg has an extended length to serve as a rod for a spinal fusion procedure on a second spinal level.

7. The dynamic spinal stabilization device of claim 1, wherein the first end comprises a hook selected from the group comprising anterior convex hooks, posterior convex hooks, anterior concave hooks, and posterior convex hooks.

8. The dynamic spinal stabilization device of claim 7, wherein the second end comprises a hook selected from the group comprising anterior convex hooks, posterior convex hooks, anterior concave hooks, and posterior convex hooks.

9. The dynamic spinal stabilization device of claim 1, wherein the second end comprises a hook selected from the group comprising anterior convex hooks, posterior convex hooks, anterior concave hooks, and posterior convex hooks.

10. The dynamic spinal stabilization device of claim 1, further comprising:
an intermediate section of the member, formed as a straight portion of the member between the second outlying loop and the second end;
a second medial loop formed as a fourth looped section of the member between the intermediate member and the second end, the second medial loop generally residing in the first plane;
a third outlying loop formed as a fifth looped section of the member between the second medial loop and the intermediate section, the third outlying loop generally residing in the second plane; and
a fourth outlying loop formed as a sixth looped section of the member between the second medial loop and the second end, the fourth outlying loop generally residing in the second plane.

11. The dynamic spinal stabilization device of claim 10, further comprising:
a second intermediate section of the member, formed as a straight portion of the member between the first outlying loop and the first end;
a third medial loop formed as a seventh looped section of the member between the intermediate member and the first end, the third medial loop generally residing in the first plane;
a fifth outlying loop formed as an eighth looped section of the member between the third medial loop and the first end, the fifth outlying loop generally residing in the second plane; and
a sixth outlying loop formed as a ninth looped section of the member between the third medial loop and the second intermediate section, the sixth outlying loop generally residing in the second plane.

12. A method of providing vertebral decompression and dynamic stabilization, the method comprising:
preparing at least a first vertebral level for placement of a dynamic spinal stabilization assembly constructed of Nitinol which comprises
a member having a cylindrical cross-section that extends from a first end to a second end;
a medial loop formed as a first looped section of the member, the medial loop generally residing in a first plane that is generally parallel to a long axis of the member and formed by the first looped section;
a first outlying loop formed as a second looped section of the member between the medial loop and the first end, the first outlying loop generally residing in a second plane that is generally parallel to the long axis of the member and generally perpendicular to the first plane formed by the medial loop;
a second outlying loop formed as a third looped section of the member between the medial loop and the second end, the second outlying loop generally residing in the second plane;
a first leg formed as a section of the member between the first outlying loop and the first end; and
a second leg formed as a section of the member between the second outlying loop and the second end;
cooling the dynamic spinal stabilization assembly to convert the Austenite structure of the Nitinol to a Martensite structure;
deforming the dynamic spinal stabilization assembly to a desired shape to ease installation;
installing the deformed dynamic spinal stabilization assembly at the at least a first vertebral level; and
warming the dynamic spinal stabilization assembly to convert the Martensite structure of the Nitinol to an Austenite structure to restore the dynamic spinal stabilization assembly construct to its original shape.

13. The method of claim 12, wherein preparing at least a first vertebral level for placement of a dynamic spinal stabilization assembly comprises preparing a vertebral lamina by grating with a laminar hook.

14. The method of claim 13, wherein installing the dynamic spinal stabilization assembly at the at least a first vertebral level comprises placement of a hook on the first end of the dynamic spinal stabilization assembly over the grated vertebral lamina.

15. The method of claim 12, wherein preparing at least a first vertebral level for placement of a dynamic spinal stabilization assembly comprises placement of pedicle screws at the standard pedicle location for spinal fusion procedures.

16. The method of claim 15, wherein installing the dynamic spinal stabilization assembly at the at least a first vertebral level comprises securing the first leg of the assembly within a channel of a pedicle screw.

17. The method of claim 16, wherein securing the first leg of the assembly within a channel of a pedicle screw further comprises placement of a protective sleeve on the first leg prior to placement in the channel of the pedicle screw.

18. The method of claim 12, further comprising performing a spinal fusion procedure on at least one adjacent vertebral level.

19. The method of claim 18, wherein the spinal fusion procedure utilizes the second leg of the dynamic spinal stabilization assembly as a rod securing the at least one adjacent vertebral level.

20. The method of claim 12, wherein cooling the dynamic spinal stabilization assembly to convert the Austenite structure of the Nitinol to a Martensite structure comprises loading the dynamic spinal stabilization assembly in saline of about 4 degrees C.

21. The method of claim 12, wherein warming the dynamic spinal stabilization assembly to convert the Martensite structure of the Nitinol to an Austenite structure to restore the dynamic spinal stabilization assembly construct to its original shape comprises exposing the dynamic spinal stabilization assembly to saline heated to from about 40 to about 45 degrees C.
